(19) United States
(12) Patent Application Publication
(10) Pub. No.: US 2022/0404285 A1
(43) Pub. Date: Dec. 22, 2022

(54) TEST KITS AND METHODS FOR IDENTIFICATION OF CANNABINOID COMPOUNDS

(71) Applicant: Compassionate Analytics Inc., Victoria (CA)

(72) Inventor: Brent Loshney, Victoria (CA)

(73) Assignee: Compassionate Analytics Inc., Victoria, BC (CA)

(21) Appl. No.: 17/767,862

(22) PCT Filed: Oct. 13, 2020
(86) PCT No.: PCT/CA2020/000116

§ 371 Date: Apr. 8, 2022

Related U.S. Application Data
(60) Provisional application No. 62/914,270, filed on Oct. 11, 2019.

(51) Int. Cl. G01N 21/78; G01N 21/29

(52) U.S. Cl. CPC G01N 21/78; G01N 21/293

(57) ABSTRACT

A kit for colorimetric identification of one or more cannabinoid compounds is disclosed. The kit includes at least two cannabinoid sensitive visualization reagents and a colour reference chart that may have at least two discernable colours, A method is also provided for colorimetric identification of one or more cannabinoid compounds in a liquid sample. The method includes contacting the liquid sample separately with at least two cannabinoid sensitive visualization reagents; allowing the at least two cannabinoid sensitive visualization reagents to develop for a defined amount of time; and comparing the resulting colour change of the at least two cannabinoid sensitive visualization reagents to a colour reference chart.

### CROSS-REFERENCE TO RELATED APPLICATION

This application claims priority to and the benefit of U.S. Provisional Patent Application Ser. No. 62/914,270 filed Oct. 11, 2019 and entitled “TEST KITS AND METHODS FOR IDENTIFICATION OF CANNABINOID COMPOUNDS,” the disclosure of which is incorporated herein by reference in its entirety for all purposes.

### FIELD

The present disclosure relates to compositions and methods for the identification of chemical compounds, more specifically compositions and methods for the colorimetric identification of cannabinoid compounds.

### BACKGROUND

Cannabinoids are a group of diverse psychoactive compounds found in Cannabis, including delta-9-tetrahydrocannabinol (THC) and cannabidiol (CBD). Other cannabinoids include cannabinol (CBN), cannabigerol (CBG), cannabidiolic acid (CBDA), cannabichromeme (CBC), cannabielsoin (CBE), cannabicyclol (CBL), and cannabicitran (CBT).

Scientific investigations have found ample evidence linking them to various pharmacological effects with potential therapeutic applications in different medical fields such as pain management, modification of behavior, reduction of anxiety and inflammation, relaxation of muscles in people with multiple sclerosis, and appetite stimulation. Cannabinoids may affect perception, mood, emotion, cognition, and motor function. Cannabis production and cannabinoids formulations have been on the rise in the last two decades due to a variety of factors, such as a change in legal landscape, a shift of social and cultural attitudes and new medical discoveries. Following this trend, medical and recreational uses of Cannabis have become increasingly popular and acceptable to the general public. For example, medical uses of Cannabis plants for pain management have been expanding quickly following recommendations by members of the medical community. In response to this trend, governmental support of research on cannabinoids have been increased to study their health effects. Cannabinoids exert their effects mainly through binding to their receptors in the brain to product neurological responses. Since the effects of cannabinoids are related to their concentrations, it is important to have a reliable system to identify cannabinoids in Cannabis products.

### SUMMARY

In an aspect of the disclosure, a kit for colorimetric identification of one or more cannabinoid compounds is disclosed. The one or more cannabinoid compounds may comprise THC, CBD or a combination of THC and CBD. The kit includes at least two cannabinoid sensitive visualization reagents and a colour reference chart. The at least two cannabinoid sensitive visualization reagents may comprise Fast Blue BB Salt or potassium hydroxide. The kit's colour reference chart may have at least two discernable colours that are viewed together to provide specificity of the colorimetric test. The kit may further comprise a developing agent to enhance the specificity of the one or more cannabinoid compounds and the developing agent may comprise a sulphuric acid solution dissolved in deionized water at a workable concentration range, such as between 1 and 2 molar (M). The kit may further comprise a developing agent to provide process consistency which may comprise isopropanol of more than 99% purity. The kit may further comprise a solvent for cannabinoid compounds to dissolve the one or more cannabinoid compounds and the solvent may comprise a carrier oil. In embodiments, the carrier oil is any of medium chained triglycerides (MCT), grapeseed oil, hemp seed oil, neem oil, olive oil, jojoba oil, or coconut oil. In embodiments, the carrier oil is any carrier oil known to a person skilled in the art. The kit may further comprise a dilution solvent to dilute the one or more cannabinoid compounds to a working range of the at least two cannabinoid sensitive visualization reagents and the dilution solvent may comprise isopropanol containing around 0.005 M potassium hydroxide. In embodiments, the dilution solvent may contain more than 0.005 M potassium hydroxide, such as more than 0.006 M, more than 0.007 M, more than 0.008 M, more than 0.009 M, or more than 0.010 M. In embodiments, the dilution solvent may contain less than 0.005 M, such as less than 0.004 M, less than 0.003 M, less than 0.002 M, or less than 0.001 M.

In another aspect of the invention, a method for colorimetric identification of one or more cannabinoid compounds in a liquid sample is provided. In embodiments, the one or more cannabinoid compounds in the method may comprise THC, CBD or a combination of THC and CBD. In other embodiments, the one or more cannabinoid compounds in the method may comprises any one or more of CBN, CBG, CBDA, CBC, CBE, CBL, and CBT.

In an aspect, the method involves contacting the liquid sample separately with at least two cannabinoid sensitive visualization reagents: allowing the at least two cannabinoid sensitive visualization reagents to develop for a defined amount of time; and comparing the resulting colour changes of the at least two cannabinoid sensitive visualization reagents to a colour reference chart. The kit's colour reference chart may have at least two discernable colours. The presence or absence of the at least two discernable colours in the at least two cannabinoid sensitive visualization reagents indicate the presence or absence of the one or more cannabinoid compounds in the liquid sample. The at least two cannabinoid sensitive visualization reagents may comprise Fast Blue BB Salt or potassium hydroxide. The method may further comprise a step of contacting the liquid sample separately with a developing agent to enhance the specificity of one or more of the cannabinoid compounds or to provide process consistency.

### BRIEF DESCRIPTION OF THE DRAWINGS

The features and advantages of the embodiments of the present disclosure will become more apparent from the detailed description set forth below when taken in conjunction with the drawings. The drawings and the associated descriptions are provided to illustrate embodiments of the disclosure and not to limit the scope of what is claimed.

FIG. 1 depicts the HPLC chromatogram and UV spectra of certified CBD and THC reference material. The retention times of CBD and THC are 3.52 and 5.39 minutes (min) respectively.

FIG. 2 depicts the HPLC chromatogram and UV spectra of the CBD formulated solution. The retention time of CBD is 3.53 min.

FIG. 3 depicts the HPLC chromatogram and UV spectra of a combination of CBD and THC formulated solution. The retention time of CBD and THC are 3.53 and 5.41 min respectively.

FIG. 4 depicts the HPLC chromatogram and UV spectra of the THC formulated solution. The retention time of THC is 5.40 min.

FIG. 5 depicts the instructions for the colorimetric identification kit.

FIG. 6 depicts the colour reference chart of the colorimetric identification kit.

FIG. 7 depicts paired vial results of tests conducted on primary formulations of a combination of THC and CBD, THC, CBD, and blank solutions with no THC or CBD.

FIG. 8 depicts replicate paired vial results of tests conducted on primary stock formulations of a combination of THC and CBD, THC, CBD, and blank solutions with no THC or CBD.

FIG. 9 depicts robustness test with variation on the primary dilution for each of the stock formulated solutions. Primary dilutions are made with 8, 10, 12 ml of dilution solvent.

FIG. 10 depicts robustness test to determine how development time affects test results. Vials are allowed to stand for 5, 10, 15 min.

FIG. 11 depicts paired vial results on formulations of THC/CBD, THC, and CBD.

FIG. 12 depicts paired vial results on formulations of THC10CBD10, THC25, and THC5CBD20.

FIG. 13 depicts a robustness test with variation on the indicator (cannabinoid sensitive visualization reagent) amount for each of the stock formulated solutions. Primary dilutions were made with 80%, 100%, and 120% of the indicator in each vial.

FIG. 14 depicts a HPLC chromatogram of CBD, CBN, THC, and THCA. Retention times of CBD and THC are 4.13 min and 6.15 min respectively.

FIG. 15 depicts a HLPC chromatogram of CBD. Retention time of CBD is 4.13.

FIG. 16 depicts a full spectrum formulation solution using an HPLC chromatogram. Retention times of CBD and THC are 4.13 min and 6.15 min respectively.

FIG. 17 depicts a full spectrum formulated solution using an HPLC chromatogram. Retention time of THC is 6.15 min.

FIG. 18 depicts colour changes when using indicator A in vials with different mass variations.

### DETAILED DESCRIPTION

### Overview of the Disclosure

The present disclosure relates to kits and related methods that are capable of providing a quantitative means to identify the presence of cannabinoids in solutions. In embodiments, the kits include indicators to identify the presence of the cannabinoids.

### Definitions

If and as used herein, in the description and the appended claims, the singular forms “a”, “an” and “the” are used interchangeably and intended to include the plural forms as well and fall within each meaning, unless the context clearly indicates otherwise. Also, as used herein, “and/or” refers to and encompasses any and all possible combinations of one or more of the listed items, as well as the lack of combinations when interpreted in the alternative (“or”).

If and as used herein, all numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about”. It also is to be understood, although not always explicitly stated, that the reagents described herein are merely exemplary and that equivalents of such are known in the art.

If and as used herein, the term “about” will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term.

If and as used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Embodiments defined by each of these transition terms are within the scope of this disclosure. Accordingly, it is intended that the methods and compositions can include additional steps and components (comprising) or alternatively including steps and compositions of no significance (consisting essentially of) or alternatively, intending only the stated method steps or compositions (consisting of).

If and as used here herein, the phrase “cannabinoid compound” refers to any compound that is found in cannabis. Examples of such compounds include delta-9-tetrahydrocannabinol (THC) and cannabidiol (CBD). Other cannabinoids include cannabinol (CBN), cannabigerol (CBG), cannabidiolic acid (CBDA), cannabichromeme (CBC), cannabielsoin (CBE), cannabicyclol (CBL), and cannabicitran (CBT).

If and as used herein, the phrase “cannabinoid sensitive visualization reagent” refers to a reagent capable of detecting the presence or absence of a cannabinoid compound in a liquid sample. The term “indicator” is used interchangeably with the phrase “cannabinoid sensitive visualization reagent.”

If and as used here, the phrase a “developing agent” refers to any agent that enhances the specificity of the one or more cannabinoid compounds in a colorimetric identification kit.

### Description of Aspects and Embodiments of the Disclosure

Test Substance

In embodiments, a kit is developed to provide a quantitative means to identify the presence of the cannabinoids THC and CBD that have been formulated in concentrated solutions. In embodiments, the kit can provide a quantitative means to identify other cannabinoids such as CBN, CBG, CBDA, CBC, CBE, CBL, and CBT.

In embodiments, the kit is designed to be a visual colorimetric detection test using paired vial indicators (cannabinoid sensitive visualization reagents) to provide specificity for solutions that contain the cannabinoids THC, CBD, or a combination of THC and CBD. In embodiments, the kit is designed to provide specificity for other cannabinoids such as any one or more of CBN, CBG, CBDA, CBC, CBE, CBL, and CBT.

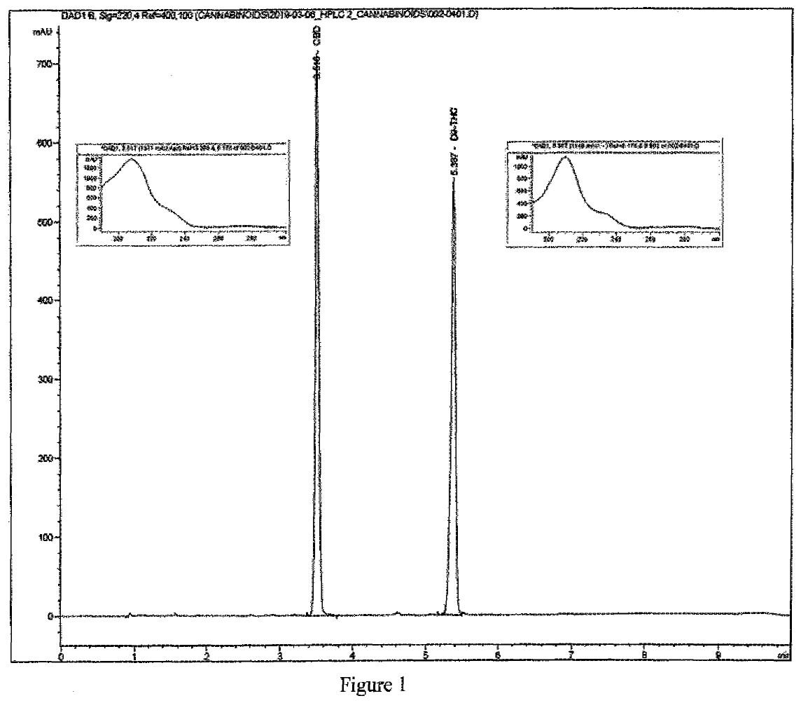

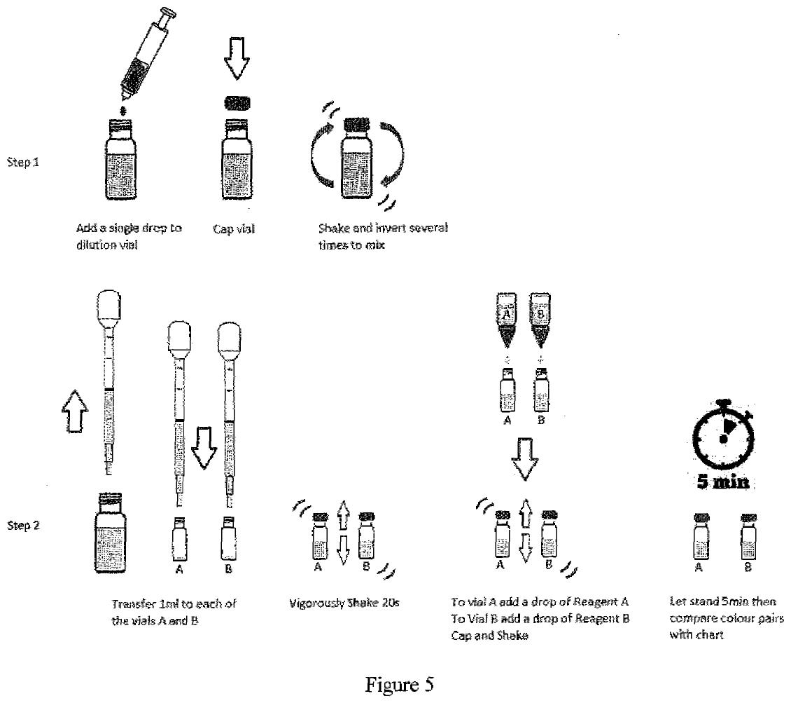

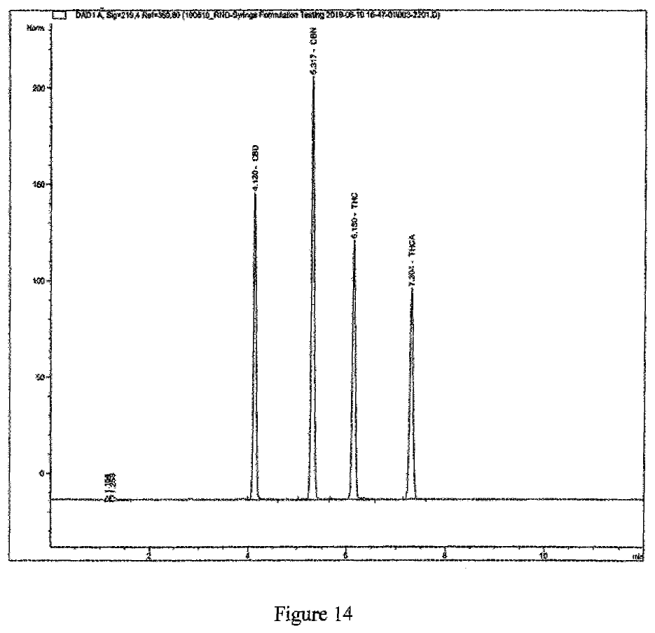

In embodiments, primary stock solutions of THC, CBD, or a combination of THC and CBD are formulated in a carrier oil of medium chained triglycerides (MCT). The carrier oil is heated. In embodiments, the carrier oil is heated to 70° C. In embodiments, the carrier oil is heated to 75° C. The required amount of active ingredients of THC, CBD or a combination of THC and CBD is added with stirring until fully dissolved. Table 1 details a formulated solution prepared for test. Additional solutions that have been prepared using Full Spectrum and Purified active materials are shown in Table 2. The active ingredients are verified and assayed by high pressure liquid chromatography (HPLC) against certified reference materials, certificates of analysis are attached in Appendix A. Primary identification was conducted by comparison of the retention times and UV spectra of THC and CBD obtained from the formulated solution with those of the reference materials as shown in FIGS. 1 to 4. Further identification of the formulated solutions is conducted by analysis using gas chromatography mass spectrometer (GC/MS). Identification is conducted by comparison of the retention times of THC and CBD obtained from the formulated solution with those of the reference materials as shown in FIGS. 14 to 17. Finished product oral solution formulations prepared in grapeseed oil and MCT oil are evaluated to verify that the colorimetric test kit is capable of qualitatively identifying the active substances present. These solutions are detailed in Table 3. It should be noted that for the finished product identification tests the primary dilution is modified as to introduce 0.25 ml of test substance to the primary dilution, using the supplied graduated dropper, in place of the single drop as described in the instructions given in FIG. 5. The modification was made because of the lower concentration of active ingredients present.

TABLE 1
Formulated Test Solutions

| Primary Stock | THC + CBD | THC | CBD | Blank |
|---|---|---|---|---|
| Active | THC/CBD | THC | CBD | none |
| Component | | | | |
| Concentration | 81.4/94.7 | 82.9 | 97.6 | ND |
| (mg/g) | | | | |

Note:
Primary stock solution of test materials used in the validation. Active components THC and CBD are solubilized in MCT oil. The formulated solutions are assayed by HPLC against certified reference materials, Cerilliant CBD lot FE01271601, THC lot FE09101501.

TABLE 2
Formulated Test Solutions

| | THC 100 + | | |
|---|---|---|---|
| Primary Stock | CBD 100 | THC 100 | CBD 200 |
| Active Component | THC/CBD | THC | CBD |
| Active Component | THC - FS | THC - FS | CBD - Purified |
| Source/(Lot) | (N0000005065) | (N0000005065) | (N0000004489) |
| | CBD - FS | | |
| | (TR031901) | | |
| Concentration | 102.5/98.0 | 99.0 | 200.7 |
| (mg/ml) | | | |

Note:
Active components THC and CBD are either Full Spectrum (FS) or Purified as indicated and are solubilized in MCT oil. The formulated solutions are assayed by HPLC against certified reference materials, Restek lot A0138786.

TABLE 3
Finished Products

| | FS OS | FS OS | FS OS |
|---|---|---|---|
| Primary Stock | THC10 CBD10 | THC25 | THC5 CBD20 |
| Active Component | THC/CBD | THC | THC/CBD |
| Finished Product/(Lot) | N0000003270 | N0000005610 | N0000002880 |
| Label Claim (mg/ml) | 10/10 | 25 | 5/20 |
| THC/CBD | | | |
| Carrier Oil | Grapeseed | Grapeseed | MCT |

Note:
Active components THC and CBD are Full Spectrum (FS) and are solubilized in Grapeseed oil or MCT oil. The finished product solutions are assayed by quality control and meet the label claim specifications.

In embodiments, the active ingredients, THC, CBD, or a combination of THC and CBD, are assayed by high pressure liquid chromatography (HPLC) against certified reference material. Primary identification of active ingredients is conducted by comparison of the retention times and UV spectra of THC and CBD obtained from the formulated solution with those of the reference materials as shown in FIGS. 1-4 and 14-17.

In embodiments, a HPLC assay of certified reference material CBD and THC exhibits a retention time of 3.52 and 5.39 min for CBD and THC respectively as demonstrated in FIG. 1.

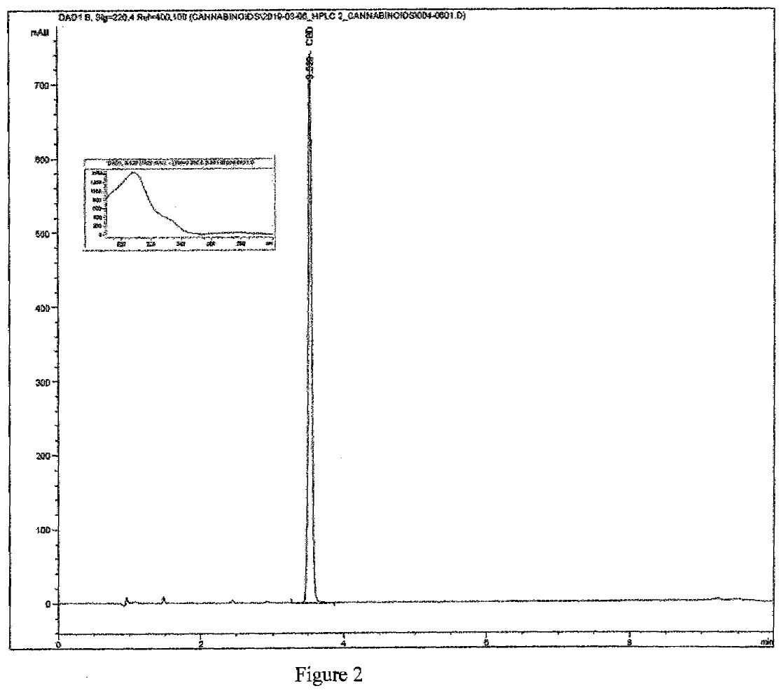

In embodiments, a HPLC assay of CBD formulated solution shows a retention time of 3.53 min, comparable to the 3.52 min for certified reference material CBD, as demonstrated in FIGS. 1 and 2.

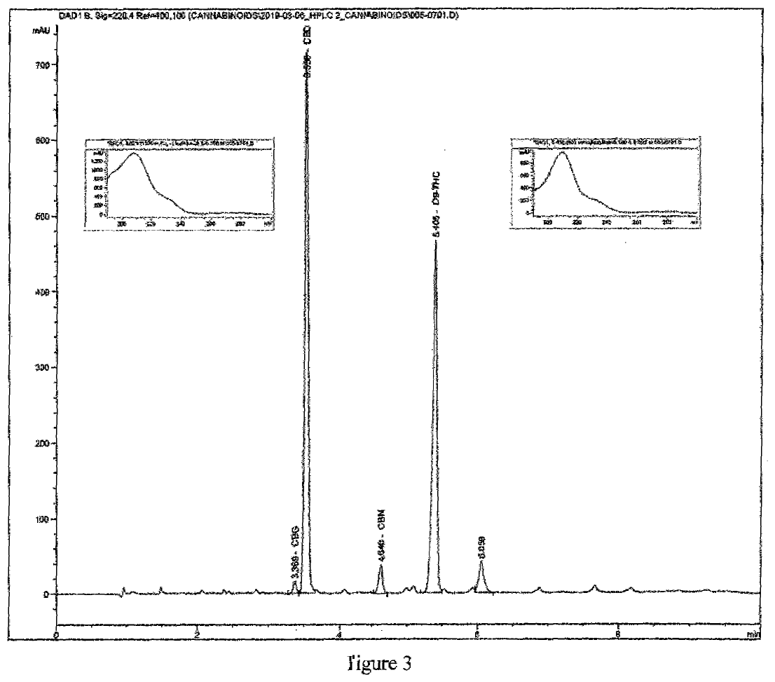

In embodiments, a HPLC assay of a combination of CBD and THC formulated solution exhibits a retention time of 3.53 and 5.41 min for CBD and THC respectively, comparable to the 3.52 and 5.39 min for certified reference material CBD and THC respectively, as demonstrated in FIGS. 1 and 3.

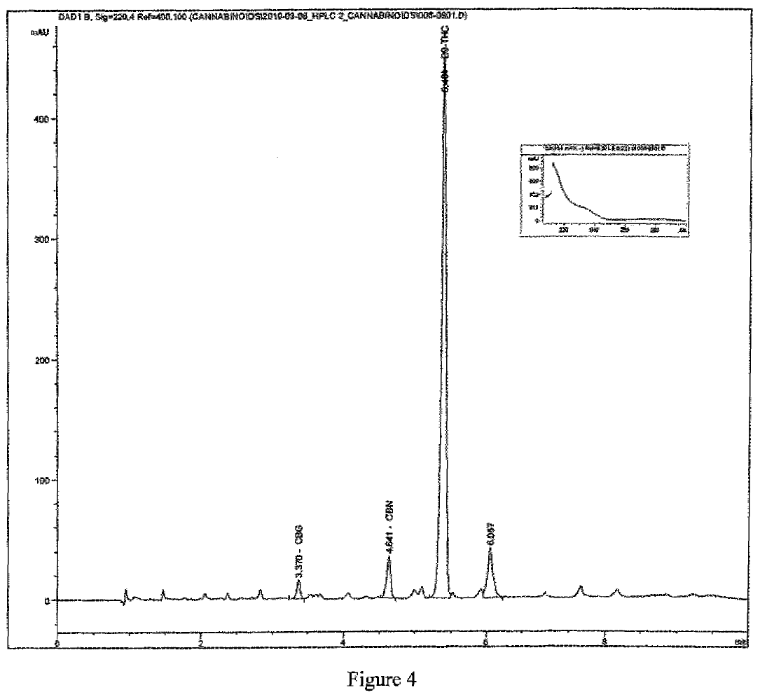

In embodiments, a HPLC assay of THC formulated solution exhibits a retention time of 5.40 min, comparable to the 5.39 min of certified reference THC, as demonstrated in FIGS. 1 and 4.

In embodiments, a HPLC assay of CBD. CBN, THC, and THCA exhibits retention times of 4.13 min and 6.15 min for CBD and THC respectively, as demonstrated in FIG. 14.

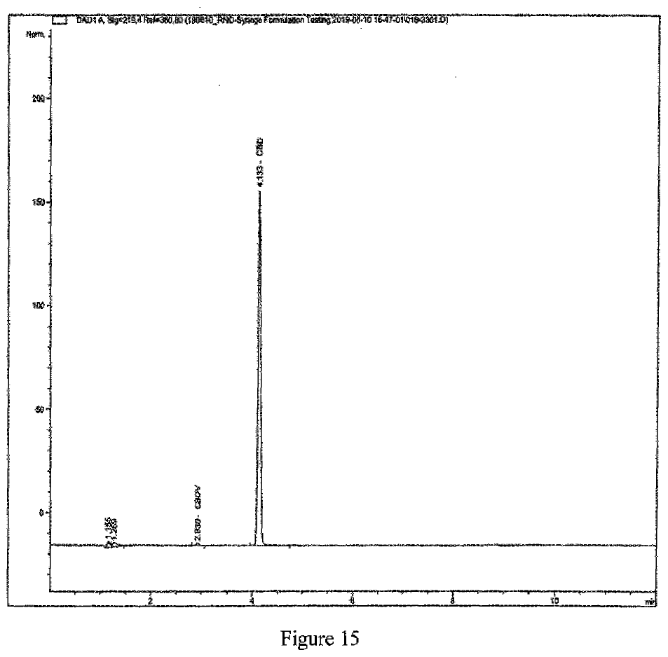

In embodiments, a HPLC assay of CBD exhibits a retention time of 4.13 for CBD, as demonstrated in FIG. 15.

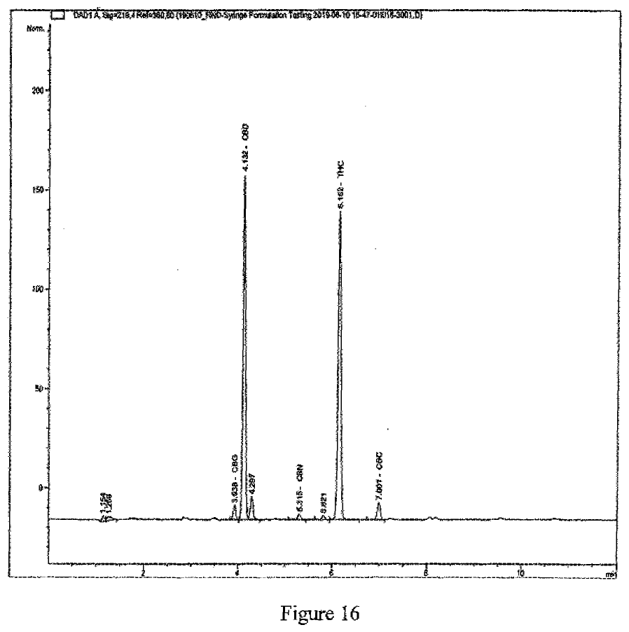

In embodiments, a HPLC assay of a full spectrum formulated solution exhibits retention times of 4.13 min and 6.15 min of CBD and THC respectively, as demonstrated in FIG. 16.

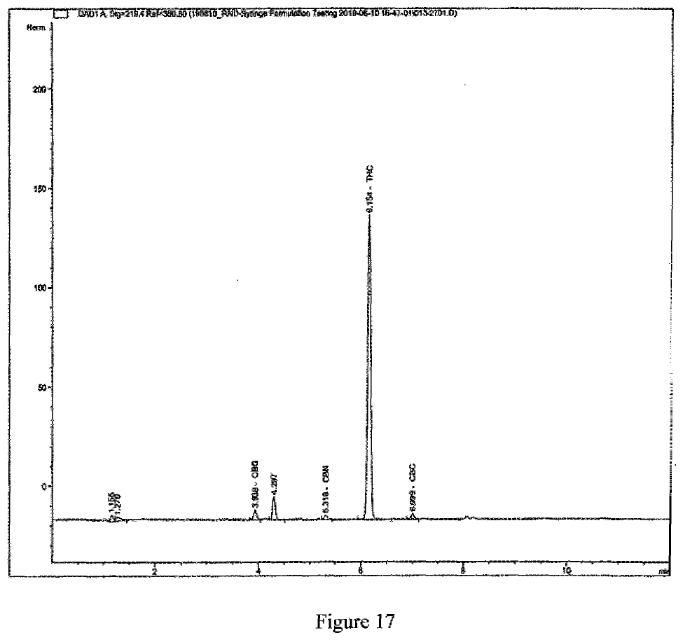

In embodiments, a full spectrum formulated solution exhibits a retention time of 6.15 min of THC, as shown in FIG. 17.

In embodiments, further identification of the formulated solutions is conducted by analysis using gas chromatography mass spectrometer (GC/MS).

Kit Instructions

The instructions for the kit are demonstrated in FIG. 5. A detailed description of the two-step instruction is as follows.

In embodiments of Step 1, the concentrations of formulated solutions of THC, CBD, or a combination of THC and CBD are reduced to the working range of the colorimetric indicator (cannabinoid sensitive visualization reagent) of the kit by adding the formulated solutions of THC, CBD, or a combination of THC and CBI) to a vial pre-filled with a dilution solvent to make a primary dilution. In embodiments, the primary dilution is about 10 milliliters (ml). In embodiments, the dilution is less than 10 ml such as less than 9 ml, less than 8 ml, less than 7 ml, less than 6 ml, less than 5 ml, less than 4 ml, less than 3 ml, less than 2 ml, or less than 1 ml. In embodiments, the primary dilution is greater than 10 ml, such as greater than 11 ml, greater than 12 ml, greater than 13 ml, greater than 14 ml, greater than 15 ml, greater than 16 ml, greater than 17 ml, greater than 18 ml, greater than 19 ml, or greater than 20 ml.

In embodiments, the formulated solution comprises THC and CBD. In embodiments, the concentration of THC is between about 0.1 mg/ml and about 15 mg/ml, for example about 1 mg/ml, about 2 mg/ml, about 3 mg/ml, about 4 mg/ml, about 5 mg/ml, about 6 mg/ml, about 7 mg/ml, about 8 mg/ml, about 9 mg/ml, about 10 mg/ml, about 11 mg/ml, about 12 mg/ml, about 13 mg/ml, about 14 mg/ml, or about 15 mg/ml. In embodiments, the concentration of CBD is between about 5 mg/ml and 25 mg/ml, for example about 5 mg/ml, about 6 mg/ml, about 7 mg/ml, about 8 mg/ml, about 9 mg/ml, about 10 mg/ml, about 11 mg/ml, about 12 mg/ml, about 13 mg/ml, about 14 mg/ml, about 15 mg/ml, about 16 mg/ml, about 17 mg/ml, about 18 mg/ml, about 19 mg/ml, about 20 mg/ml, about 21 mg/ml, about 22 mg/ml, about 23 mg/ml, about 24 mg/ml, or about 25 mg/ml.

In embodiments, the formulated solution of THC and CBD comprises a concentration of THC that is between about 3 mg/ml and 7 mg/ml, and a concentration of CBD is between about 18 mg/ml and 22 mg/ml. For example, the concentration of THC is about 3 mg/ml, about 4 mg/ml, about 5 mg/ml, about 6 mg/ml, or about 7 mg/ml and the concentration of CBD is about 18 mg/ml, about 19 mg/ml, about 20 mg/ml, about 21 mg/ml, or about 22 mg/ml. In embodiments, the concentration of THC is about 5 mg/ml and the concentration of CBD is about 20 mg/ml.

In embodiments, the formulated solution of THC and CBD comprises a concentration of THC that is between about 8 mg/ml and 12 mg/ml, and a concentration of CBD that is between about 8 mg/ml and 12 mg/ml. For example, the concentration of THC is about 8 mg/ml, about 9 mg/ml, about 10 mg/ml, about 11 mg/ml, or about 12 mg/ml, and the concentration of CBD is about 8 mg/ml, about 9 mg/ml, about 10 mg/ml, about 11 mg/ml, or about 12 mg/ml. In embodiments, the concentration of THC is about 10 mg/ml and the concentration of CBD is about 10 mg/ml.

In embodiments, the formulated solution comprises THC. In embodiments, the concentration of THC is between about 20 mg/ml and about 30 mg/ml. For example, the concentration of THC is about 20 mg/ml, about 21 mg/ml, about 22 mg/ml, about 23 mg/ml, about 24 mg/ml, about 25 mg/ml, about 26 mg/ml, about 27 mg/ml, about 28 mg/ml, about 29 mg/ml, or about 30 mg/ml. In embodiments, the concentration of THC is about 25 mg/ml.

In embodiments, between about 0.15 ml and 0.35 ml of the formulated solution of THC, CBD, or a combination of THC and CBD is added to between about 5 ml and 15 ml of diluent solvent. In embodiments, the amount of formulated solution is about 0.15 ml, about 0.16 ml, about 0.17 ml, about 0.18 ml, about 0.19 ml, about 0.20 ml, about 0.21 ml, about 0.22 ml, about 0.23 ml, about 0.24 ml, or about 0.25 ml. In embodiments, the amount of diluent solvent is about 5 ml, about 6 ml, about 7 ml, about 8 ml, about 9 ml, about 10 ml, about 11 ml, about 12 ml, about 13 ml, about 14 ml, or about 15 ml. In embodiments, the amount of formulated solution is about 0.25 ml and the amount of diluent solvent is about 10 ml.

In embodiments, the dilution solvent will provide a basic media for the Fast Blue BB Salt indicator to react with the active compounds in the primary solution. In embodiments, other types of indicators could be used such as those listed in Table 4.

TABLE 4
Types of Indicators

| Name | CAS | Synonyms | MW | Formula | Structure |
|---|---|---|---|---|---|
| Fast Blue B Salt | 14263-94-6 | o-Dianisidine bis(diazotized) zinc double salt, Azoic Diazo No. 48, DBB, Diazo Blue B, Naphthanil Diazo | 475.47 | C₁₄H₁₂N₄O₂Cl₂•ZnCl₂ | 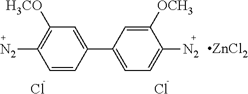 |
| | | Blue B | | | |
| Fast Blue BB Salt hemi(zinc chloride) salt | 5486-84-0 | 4-Amino-2,5- diethoxybenzanilide diazotated zinc double salt, 4-Benzoylamino- 2,5-diethoxy- benzenediazonium chloride hemi(zinc chloride) salt | 415.94 | C₁₇H₁₈N₃O₃Cl•1/2 ZnCl₂ | 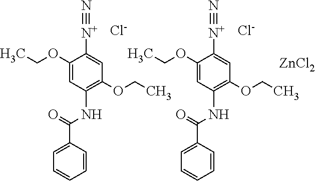 |
| Fast Blue RR | 6268-05-09 | 4-Benzoylamino-2,5- dimethoxyaniline, Azoic Diazo No. 24 | 272.3 | C₁₅H₁₆N₂O₃ | 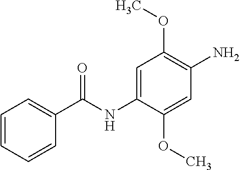 [SMILES: COc1cc(NC(=O)c2ccccc2)c(OC)cc1N] |
| Fast Blue RR Salt | 14726-29-5 | 4-Benzoylamino-2,5- dimethoxy- benzenediazonium chloride hemi(zinc chloride) salt, Azoic Diazo No. 24 | 387.89 | C₁₅H₁₄ClN₃O₃•1/2 ZnCl₂ | 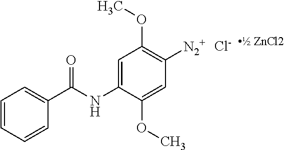 |
| N-(4-Amino- 2,5- diethoxyphenyl) benzamide | 120-00-3 | 4′-Amino-2′,5′- diethoxybenzanilide, Azoic Diazo No. 20, Fast blue BB | 300.35 | C₁₇H₂₀N₂O₃ | 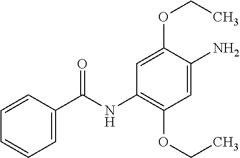 [SMILES: CCOc1cc(NC(=O)c2ccccc2)c(OCC)cc1N] |

In embodiments, the dilution solvent is made by dissolving finely ground potassium hydroxide in isopropanol to a final concentration of about 0.005 M potassium hydroxide. In embodiments, the final concentration of potassium hydroxide is less than 0.005 M such as less than 0.005 M, less than 0.003 M, less than 0.002 M, or less than 0.001 M. In embodiments, the final concentration of potassium hydroxide is greater than 0.005 M such as greater than 0.006 M, greater than 0.007 M, greater than 0.008 M, greater than 0.009 M, or greater than 0.010 M. In embodiments, methanol or ethanol or a lower alcohol is used as a substitute for isopropanol in the dilution solvent. In embodiments, other alcohols may be use in the dilution solvent. In other embodiments, sodium hydroxide or another base is used as a substitute for potassium hydroxide in the dilution solvent. In embodiments other hydroxides may be used in the dilution solvent.

In embodiments of Step 1, the vial containing the primary solutions of THC, CBD, or a combination of THC and CBD is capped and then shaken and inverted several times to mix.

In embodiments of Step 2, 1 ml of the primary dilution is transferred to each of the two vials, A and B.

In embodiments of Step 2, vial A contains 5 milligrams (mg) of colorimetric indicator A, a mixture of Fast Blue BB Salt powder with table sugar in a ratio of 1 to 9 by weight.

In embodiments of Step 2, vial B contains 5 mg of colorimetric indicator B, potassium hydroxide solid prepared by lightly grinding and mixing bulk potassium hydroxide until it is well powderized. In other embodiments, sodium hydroxide or another base is used as a substitute for potassium hydroxide in the colorimetric indicator B.

In embodiments of Step 2, vials A and B are capped and vigorously shaken for 20 seconds.

In embodiments of Step 2, 0.031 ml of the Developing Agent A, 1.7 M sulphuric acid solution dissolved in deionized water, is added to vial A to further enhance the specificity of the THC compound. In embodiments of Step 2, 0.031 ml of the Developing Agent B, isopropanol, is added to vial B to provide process consistency with vial A with no effect on the final colorimetric reaction. In other embodiments, hydrochloric acid or another acid is used as a substitute for sulphuric acid in the Developing Agent A. In other embodiments, methanol or ethanol or a lower alcohol is used as a substitute for isopropanol in the Developing Agent B.

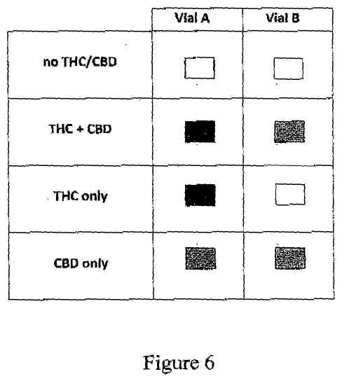

In embodiments of Step 2, vial A and B are allowed to stand for 5 min to develop the final colour for comparison to a reference chart shown in FIG. 6.

In embodiments, the colorimetric reaction can take place in a liquid solution or on a colorimetric test strip. Further, in embodiments, the visualization of the color change can be visual (e.g., by naked eye) or through ue of an instrument such as a spectrophotometer.

Validation Items

In embodiments, validation of the kit is conducted for specificity, accuracy, precision and robustness. Specificity is assessed by evaluating the kit's ability to positively identify the presence or absence of THC or CBD in the formulation. Accuracy is assessed by qualitatively comparing the colorimetric test result to a reference colour chart. Precision is assessed by evaluating whether the kit can provide the same qualitative colorimetric result for multiple iterations. Robustness is assessed by evaluating how deliberate changes to the method can affect the final result. Specificity, accuracy, and precision are also evaluated for the products made at Tilray Canada Inc. to provide a secondary measure of confidence that the validation parameters are met for THC and CBD identification in the presence of Full Spectrum and Purified extracts as well as finished produces prepared in grapeseed and MCT oil carriers.

Specificity

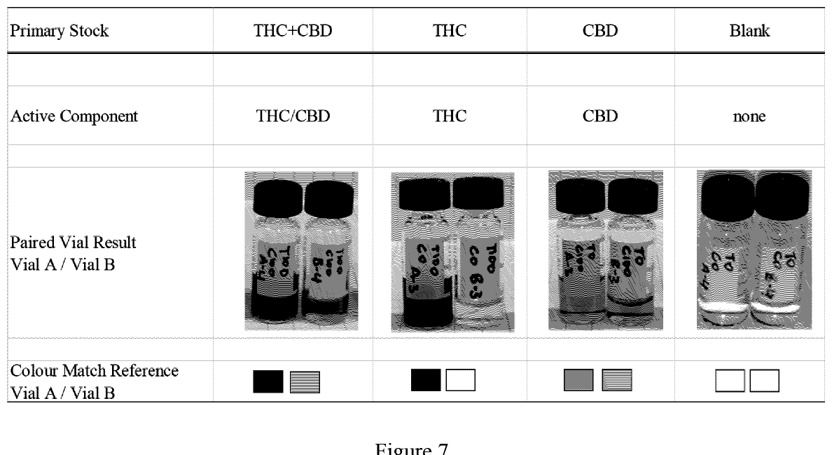

In embodiments, tests to confirm specificity are conducted by analyzing the formulated stock solution made from Tables 1, 2, and 3. The paired vial colorimetric test requires that each vial of the pair to be viewed together and the assessment made in relation to the colour chart in FIG. 6. The test is conducted with each of the stock solutions and photos are collected as confirmation. The results are given in FIGS. 7, 11, and 12. Paired vial visual results clearly show a discernable colour difference for each of the pair vial test solution indicating specificity is achieved. Specifically, Vial A is of a different colour than Vial B, in each of the formulations. Visual results of FIG. 7 also correlate with reference chart (FIG. 6) satisfying the accuracy requirement. Specifically, for example, Vial A the THC/CBD formulation in FIG. 7 is the same colour as shown in Vial A of the THC+CBD formulation in the reference chart (FIG. 6). Similarly, the colour of each of the Vials within each of the formulations of FIG. 7 matches its counterpart reference colour in FIG. 6.

Accuracy

In embodiments, the determination of the formulated test solutions by the paired vial colorimetric test provides an accurate means of identity as shown by the results collected in FIGS. 7, 11, and 12. An accurate determination between solutions that contain THC, CBD or a combination of THC and CBD is achieved, and the positive result is indicated by comparison to the colour chart in FIG. 6. A negative result for formulations that do not contain these active components can also be accurately determined as the indicators are not reacting resulting in no colour change. By these measures the test can be considered accurate.

Precision

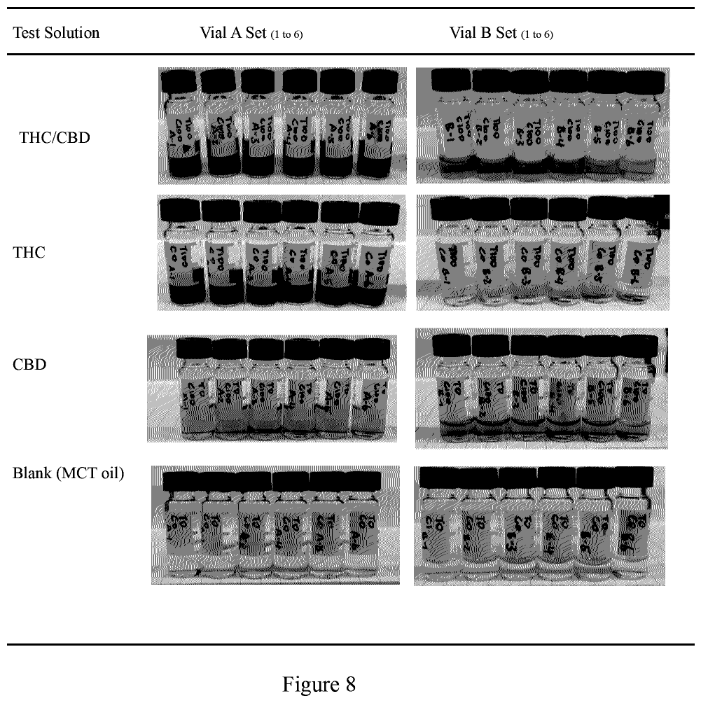

In embodiments, the paired vial colorimetric test is verified for precision by conducting the identification test on the stock formulations made in Table 1 six (6) times. The Tilray Canada Inc. formulations are shown in Table 2 (3) times and the Tilray Canada Inc. finished product formulations are shown in Table 3 three (3) times. The colour for each of the replicates is then compared to each other to determine the reproducibility of the test. The colour for each of the 6 replicates is then compared to each other to determine how reproducible the test is. FIGS. 8, 11, and 12 give the results of the test. For each of the stock formulations the vial sets have no discernable difference in colour visually and the test can be verified as repeatable. Specifically, each vial of Vial A Set of each formulation is the same colour, and each vial of Vial B Set of each formulation is the same colour. Moreover, the colour of each of the vials within Vial A Set within each formulation matches its counterpart reference colour in FIG. 6, and the colour of each of the vials with Vial B Set within each formulation matches its counterpart reference colour in FIG. 6.

Robustness

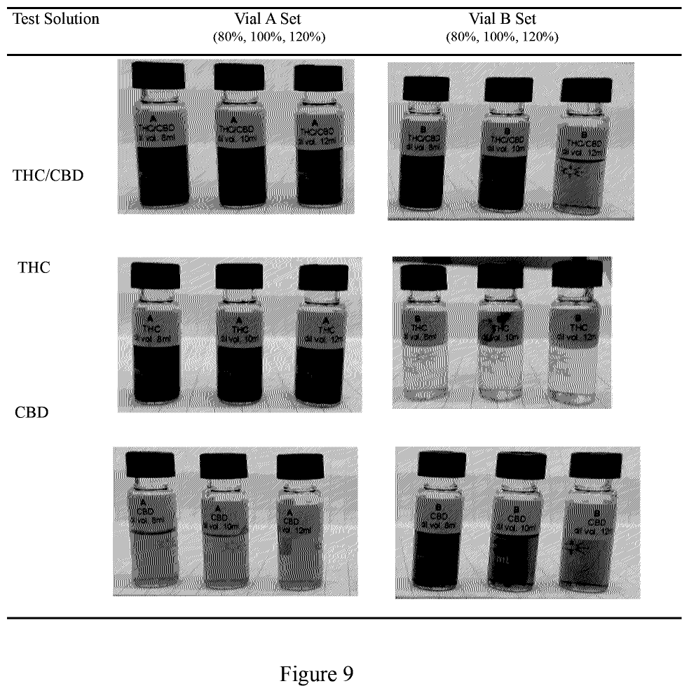

In embodiments, variations in the primary dilution volume are changed by an increase or decrease of 20% to determine what the impact of dilution of the primary stock solution is. In embodiments, primary stock solutions are diluted with 8 ml (80%), 10 ml (100%) and 12 ml (120%) of the dilution solvent, 0.005 M potassium hydroxide in isopropanol, and the tests are carried through using the normal test procedure. The results of the tests are given in FIG. 9. The 12-ml test solutions for a combination of THC and CBD formulation showed colours which are slightly more transparent than the default test for both the Vial A and Vial B solutions. Although this change is noticeable it is evident what colour the vial is and how it compares to the colour chart in FIG. 6. Moreover, despite the slight colour change across the different concentrations in FIG. 9, each of the vials in Vial A Set of each formulation can be matched to its counterpart reference colour in FIG. 6, and each of the vials and Vial B Set of each formulation can be matched to its counterpart reference colour in FIG. 6.

The test solutions for the THC formulation showed no difference in colour variance for the different dilution volumes for either of Vial A or Vial B sets. The test solutions for the CBD formulation showed no colour variance for the Vial A set. The Vial B set did show a lighter trend with the more dilute solution, again this does not affect the visual determination of the colorimetric test. It can be concluded that primary stock solutions that have higher or lower concentrations of the stock formulation will still provide accurate determination of results. In other embodiments, methanol or ethanol or a lower alcohol is used as a substitute for isopropanol in the dilution solvent. In other embodiments, sodium hydroxide or another base is used as a substitute for potassium hydroxide in the dilution solvent.

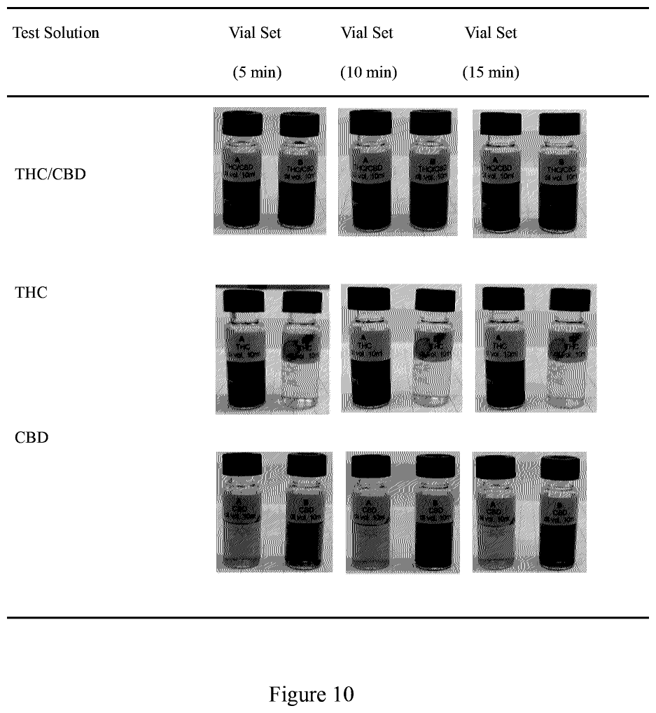

In embodiments, variation of the time of colorimetric development is investigated. Primary solutions are prepared in accordance with the test procedure from the stock formulations. The primary solutions are added to the respective indicator vials (Vials A and B) and the corresponding development reagent is added. The vials are allowed to stand for the prescribed amount of time, 5 minutes. The vials are left to stand for an additional 5 and 10 min documented with photos for each time point. The data for this study is collected and tabulated in FIG. 10. No visual colorimetric change can be detected from the 5-minute default development time indicating that the developed solutions are stable for the given time necessary to make the colorimetric determination.

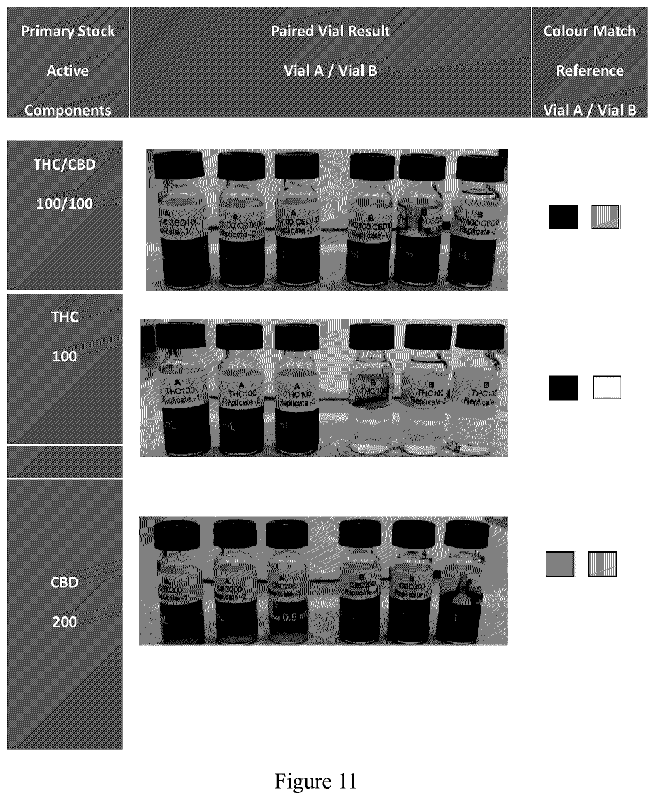

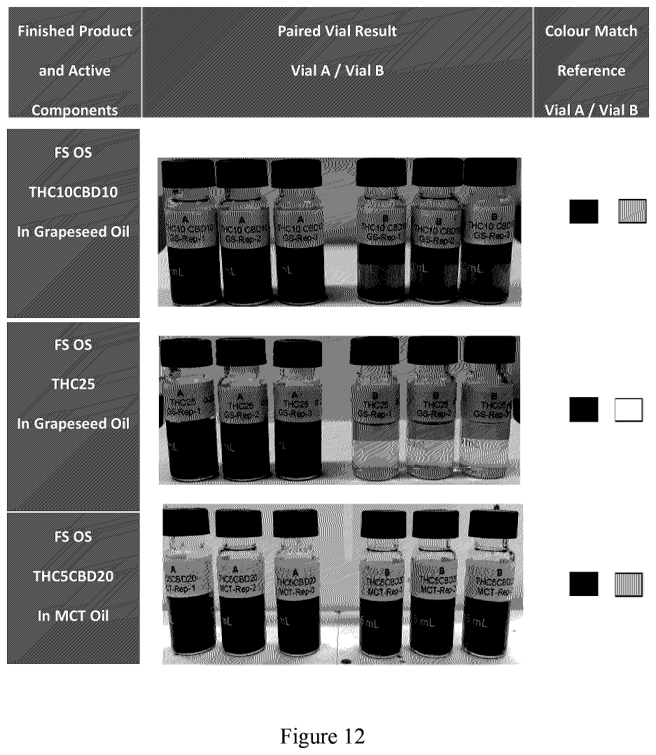

Paired vial results were also conducted on the Tilray Canada Inc. formulations. Paired visual results were unique and satisfied specificity requirements. Visual results were correlated with a reference chart satisfying the accuracy requirement. As shown in FIGS. 11 and 12, precision was achieved with no discernable difference in colour as seen visually for each triplicate vial set. Specifically, as shown in each formulation (i.e. each row) of FIGS. 11 and 12, the vials in the vial A triplicate are all the same colour, and the vials in the vial B triplicate are all the same colour.

In embodiments, variation in the amount of indicator (cannabinoid sensitive visualization reagent) present in each vial is changed by ±20% to determine the impact on the developed colorimetric result. In embodiments, the amount of indicator present in each vial is changed by greater than 20%, for example, greater than 21%, greater than 22%, greater than 23%, greater than 24%, greater than 25%, greater than 26%, greater than 27%, greater than 28%, greater than 29%, or greater than 30%.

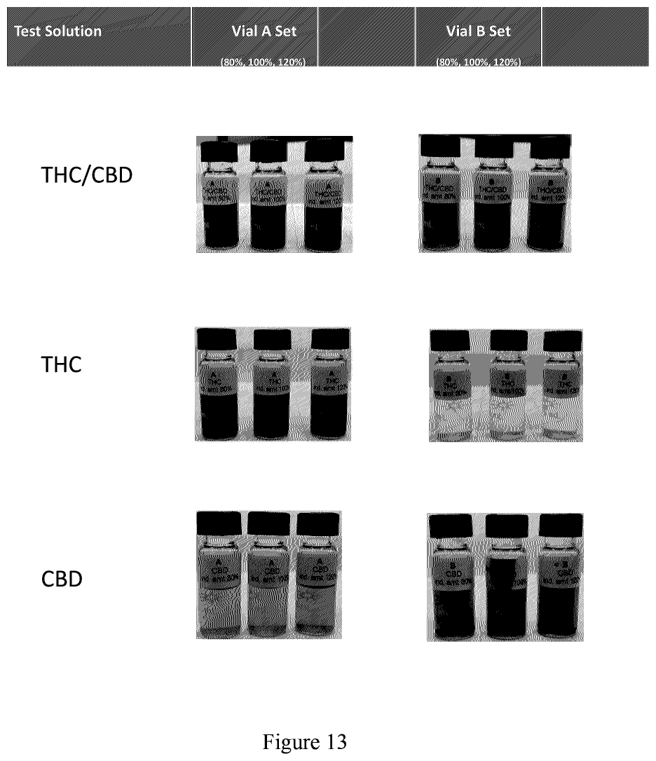

In embodiments, primary solutions are prepared in accordance with the test procedure from the stock formulations. The primary solutions are added to the respective indicator vials (A and B) with varying amount of indicator present-80%, 100% and 120% of the required amount. In embodiments, the amount of indicator present is less than 80%. In embodiments, the amount of indicator is greater than 120%. The test is completed as per the procedure and the vial colours for each set are tabulated for examination in FIG. 13. The variation of the indicator in this range did not have a discernable effect on the colour or intensity as compared to the vials containing the prescribed amount of indicator. As a result, each of the vials in Vial A Set of each formulation can be matched to its counterpart colour in FIG. 6, an each of the vials in Vial B Set of each formulation can be match to its counterpart colour in FIG. 6.

### EXAMPLES

### Example 1: Preparation of the Dilution Solvent

Approximately 0.5 liter (L) of isopropanol is added to a 1 L bottle. Then, 0.28 gram (g) of finely ground potassium hydroxide is added into the isopropanol followed by sonication to help it dissolve. More isopropanol is added to make a final volume of 1 L of 0.005 M potassium hydroxide. The bottle is capped and inverted several times to mix the solution. The integrity of this solution is estimated to be viable for 6 months. The isopropanol's CAS registry number is 67-63-0 with a molecular weight of 60.1 and a purity of more than 99%. The potassium hydroxide's CAS registry number is 1310-58-3 with a molecular weight of 56.1 and a purity of more than 99%.

### Example 2: Preparation of Fast Blue BB Salt Indicator (Indicator A)

1 g of Fast Blue BB Salt powder and 9 g of fine particulate table sugar are added to a mortar and pestle. The mixture is lightly ground and mixed with the mortar and pestle until well blended. The mixture is then transferred to an amber glass bottle tightly capped for storage at ambient temperature away from light. Indicator A acts as the colorimetric indicator for the presence of cannabinoids. Dilution with sugar is necessary to facilitate accurate dispensing of the final blended mixture. The Fast Blue BB Salt's CAS registry number is 5486-84-0 with a molecular weight of 415.94 and a purity of more than 99%.

### Example 3: Preparation of Potassium Hydroxide (Indicator B)

10 g of bulk potassium hydroxide is added to a mortar and pestle followed by lightly grinding and mixing until the potassium hydroxide is well powderized. The potassium hydroxide powder is transferred to an amber glass bottle for storage at ambient temperature in a desiccator. Its integrity is estimated to be viable for 6 months. The potassium hydroxide's CAS registry number is 1310-58-3 with a molecular weight of 56.1 and a purity of more than 99%.

### Example 4: Preparation of Sulphuric Acid Reagent (Developing Agent A)

Approximately 0.5 L of deionized water and 93 ml of concentrated sulphuric acid are added to a 1 L bottle. The solution is gently swirled to mix and then more deionized water is added to make to a final volume of 1 L. The bottle is capped and mixed by inverting several times. The integrity of this solution is estimated to be viable for 6 months. The amount of Developing Agent A required for the kit is 2 ml. The sulphuric acid's CAS registry number is 7664-93-9 with a molecular weight of 98.1 and a purity of more than 98%. The deionized water's CAS registry number is 7732-18-5 with a molecular weight of 18.02 and a purity of more than 99%.

### Example 5: Preparation of Isopropanol Reagent (Developing Agent B)

The isopropanol is dispensed directly into the dropper bottle. The integrity of this solution is estimated to be viable for 3 years. The amount of Developing Agent B required for the kit is 2 ml. The isopropanol's CAS registry number is 67-63-0 with a molecular weight of 60.1 and a purity of more than 99%.

The present disclosure also makes reference and incorporates by reference Appendix A-1 and B-1.

### Example 6: Indicator A-Mass Variation Observations

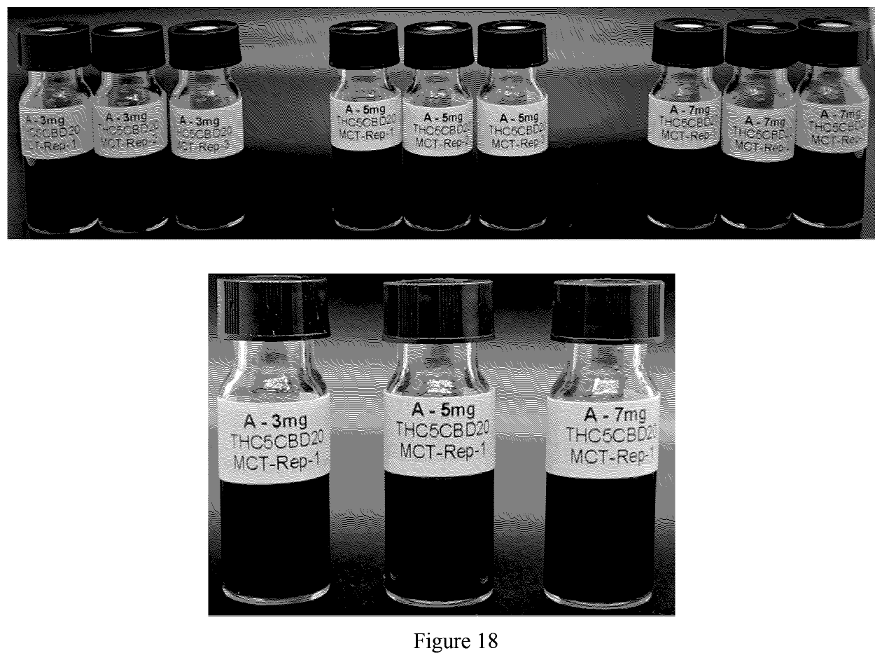

Indicator A was weighed into vials in triplicate at levels of 3 mg, 5 mg, and 7 mg. A test sample of T5C20 (the lowest and most difficult matrix for the THC indicator test) was prepared as per the ID test instructions. An amount of 0.25 ml of the T5C20 test sample was transferred to the diluent vial containing 10 ml of 0.005 M KOH. The solution was then mixed. An amount of 1 ml of the solution was then transferred to each of the indicator vials and shaken vigorously for 20 seconds. One drop of the developing agent A was added to the solution after which the vials were capped and mixed again. After 5 min photos were taken to record the results. As shown in FIG. 18, all of the test vials produced an acceptable colour change in order to be identified by the qualitative test.

What is claimed is:

1. A kit for colorimetric identification of one or more cannabinoid compounds, comprising:
at least two cannabinoid sensitive visualization reagents; and
a colour reference chart.

2. The kit of claim 1, wherein the one or more cannabinoid compounds comprise THC.

3. The kit of claim 1, wherein the one or more cannabinoid compounds comprise CBD.

4. The kit of claim 1, wherein the one or more cannabinoid compounds comprise a combination of THC and CBD.

5. The kit of claim 1, wherein the at least two cannabinoid sensitive visualization reagents comprise Fast Blue BB Salt.

6. The kit of claim 1, wherein the at least two cannabinoid sensitive visualization reagents comprise potassium hydroxide.

7. The kit of claim 1, wherein the colour reference chart comprises at least two discernable colours.

8. The kit of claim 1, further comprising a developing agent to enhance the specificity of one or more of the cannabinoid compounds.

9. The kit of the claim 8, further comprising a developing agent to provide process consistency.

10. The kit of the claim 8, wherein the developing agent comprises sulphuric acid solution dissolved in deionized water at a workable concentration range.

11. The kit of the claim 10, wherein the workable concentration range is between 1 M and 2 M.

12. The kit of the claim 9, wherein the developing agent comprises isopropanol of more than 99% purity.

13. The kit of claim 1, further comprising a solvent for cannabinoid compounds to dissolve the one or more cannabinoid compounds.

14. The kit of claim 13, wherein the solvent comprises a carrier oil of medium chained triglycerides.

15. The kit of claim 1, further comprising a dilution solvent to dilute the one or more cannabinoid compounds to a working range of the at least two cannabinoid sensitive visualization reagents.

16. The kit of claim 15, wherein the dilution solvent comprises isopropanol containing 0.005 M potassium hydroxide.

17. A method for colorimetric identification of one or more cannabinoid compounds in a liquid sample, comprising:
contacting the liquid sample separately with at least two cannabinoid sensitive visualization reagents;
allowing the at least two cannabinoid sensitive visualization reagents to develop for a defined amount of time; and
comparing the resulting colour change of the at least two cannabinoid sensitive visualization reagents to a colour reference chart.

18. The method of claim 17, wherein the one or more cannabinoid compounds comprise THC.

19. The method of claim 17, wherein the one or more cannabinoid compounds comprise CBD.

20. The method of claim 17, wherein the one or more cannabinoid compounds comprise a combination of THC and CBD.

21. The method of claim 17, wherein the at least two cannabinoid sensitive visualization reagents comprise Fast Blue BB Salt.

22. The method of claim 17, wherein the at least two cannabinoid sensitive visualization reagents comprise potassium hydroxide.

23. The method of the claim 17, further comprising a step of contacting the liquid sample separately with a developing agent to enhance the specificity of one or more of the cannabinoid compounds or to provide process consistency.

24. A method, comprising:
transferring a solution comprising one or more cannabinoid compounds to first and second vials, wherein the first vial contains a first colorimetric indicator and the second vial contains a second colorimetric indicator;
mixing the first and second vials; and
incubating the first and second vials for a pre-determined time to develop a final colour that can be compared to a reference chart.

25. The method of claim 24, wherein the first colorimetric indicator comprises Fast Blue BB salt and table sugar.

26. The method of claim 24, wherein the second calorimetric indicator comprises a hydroxide, and preferably a potassium hydroxide or sodium hydroxide.

27. The method of claim 24, further comprising adding a first developing agent to the first vial and a second developing agent to the second vial.

28. The method of claim 27, wherein the first developing agent comprises a hydrochloric acid solution or a sulphric acid solution.

29. The method of claim 27, wherein the second developing agent comprises methanol, ethanol, or isopropanol.

30. The method of claim 24, wherein the at least one or more cannabinoid compounds comprise THC, CBD, or a combination of THC and CBD.
